(19) United States
(12) Patent Application Publication
(10) Pub. No.: US 2024/0197528 A1
(43) Pub. Date: Jun. 20, 2024

(54) INSTRUMENT FOR DELAMINATING RETINAL MEMBRANES

(71) Applicant: Alcon Inc., Fribourg (CH)

(72) Inventors: Reto Grüebler, Greifensee (CH); Luca Palmerini, Basel (CH)

(21) Appl. No.: 18/535,263

(22) Filed: Dec. 11, 2023

Related U.S. Application Data
(60) Provisional application No. 63/387,335, filed on Dec. 14, 2022.

(51) Int. Cl. A61F 9/007

(52) U.S. Cl. CPC A61F 9/00736; A61B 2017/320044

(57) ABSTRACT

An ophthalmic surgical instrument for delaminating a retinal membrane includes a handpiece and an actuator mounted on the handpiece. An outer tube has a proximal end mounted to the handpiece. A dissection spatula is extendable outwardly relative to a distal end of the outer tube responsive to movement of the actuator, the dissection spatula defining a channel connected to an opening proximate a distal edge of the dissection spatula. A dispensing system is in fluid communication with the channel. The dispensing system may include a reservoir within the handpiece. The fluid may be dyed blue to enhance visibility of a delaminated portion of the retinal membrane.

### BACKGROUND

The internal limiting membrane (ILM) is a thin transparent membrane positioned between the vitreous and the retina of the eye. The ILM plays a role during the formation of the eye but is not required for the proper function of an adult eye. The ILM may pull at the retina and cause conditions such as macular holes, macular pucker, vitreo-macular traction syndrome, diabetic macular edema, and cystoid macular edema secondary to inflammation or venous occlusive diseases and other conditions. An epiretinal membrane (ERM) is a membrane that may form over the retina in response to damage to the retina, such as due to posterior vitreous detachment.

The ILM or ERM may need to be peeled away from the retina to prevent damage to the retina. Peeling of the ILM or ERM may also be required in preparation for surgical procedures performed on the retina. To peel the ILM or ERM, a surgical instrument is inserted through a cannula within the patient's eye globe. Forceps or a specialized scraper are extended from the instrument and used to raise a flap in the ILM or ERM. The flap is then grasped by the forceps and the ILM or ERM is peeled away from the retina using a circular motion. Excess force on the forceps may result in piercing of the retina.

It would be an advancement in the art to reduce the risk of retinal damage resulting from membrane peeling.

### SUMMARY

The present disclosure relates generally a structure for delaminating a retinal membrane.

Certain aspects provide an ophthalmic surgical instrument for delaminating a retinal membrane, the instrument including a handpiece and an actuator mounted on the handpiece. An outer tube has a proximal end mounted to the handpiece. A dissection spatula is extendable outwardly relative to a distal end of the outer tube responsive to movement of the actuator, the dissection spatula defining a channel connected to an opening proximate a distal edge of the dissection spatula. A dispensing system is in fluid communication with the channel.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure.

FIG. 1A is an isometric view of a surgical instrument having a dissection spatula with an integrated channel, in accordance with certain embodiments.

FIG. 1B is a side view of the dissection spatula, in accordance with certain embodiments.

FIGS. 2A to 2C are cross-sectional views showing delamination using the dissection spatula with the integrated channel, in accordance with certain embodiments.

FIG. 3 is an isometric view showing an ILM being peeled using forceps following delamination, in accordance with certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide a surgical instrument for delaminating a membrane from a patient's retina. Note that, herein, a distal end of a component refers to the end that is closer to a patient's body while the proximal end of the component refers to the end that is facing away from the patient's body or in proximity to, for example, the handpiece of the surgical instrument.

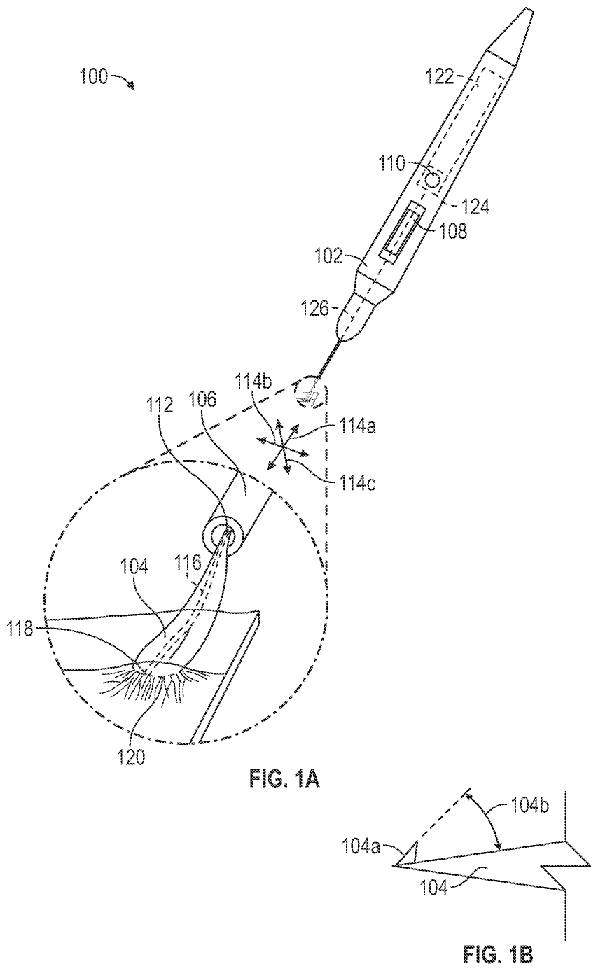

FIG. 1A illustrates a surgical instrument 100, in accordance with certain embodiments, including a handpiece 102 that is sized and contoured to be grasped by a hand of a surgeon performing an ophthalmic surgical procedure such as peeling of a membrane from a retina of a patient's eye, such as an ILM or ERM. The surgical instrument 100 may be used to delaminate the membrane from the retina after which the membrane may be removed using forceps or other grasping instrument.

A dissection spatula 104 is extendable from a distal end of an outer tube 106 connected to the handpiece 102. The proximal end of the outer tube 106 is connected to the handpiece 102. The handpiece 102 may have one or more manual control structures mounted thereto. In the embodiment of FIG. 1A, the manual control structures include a slider 108 and a button 110. The manual control structures shown are exemplary only and other manual control structures may also be used. In particular, the slider 108 may be used to control extension of the dissection spatula 104 relative to the outer tube and may be replaced with a deformable basket.

The dissection spatula 104 is connected to and defined around a hollow rod 112 extending through the outer tube 106. In a first implementation, the hollow rod 112 is fixed relative to the handpiece 102 whereas the outer tube 106 is slidable relative to the handpiece 102 and is coupled to the slider 108 to be actuated thereby. In a second implementation, the hollow rod 112 is coupled to the slider 108 and is actuated thereby whereas the outer tube 106 is fixed relative to the handpiece 102.

A longitudinal direction 114a may be defined as parallel to and collinear with the axis of symmetry of the outer tube. A transverse direction 114b may be defined as perpendicular to the longitudinal direction 114a and a vertical direction 114c may be defined as perpendicular to the longitudinal direction 114a and the transverse direction 114b.

The dissection spatula 104 extends distally from the hollow rod 112 and increases in width in the transverse direction 114b such that the dissection spatula 104 at its widest point in the transverse direction 114b is many times greater than the thickness of the dissection spatula 104 perpendicular to the transverse direction 114b, such as greater than 2, 5, 10, 20, or 30 times the thickness. The width of the dissection spatula 104 at its widest point may also be greater than the inner diameter of the outer tube 106, such as between 1.1 and 2 times the inner diameter. Accordingly, when retracted within the outer tube 106, the dissection spatula 104 may curl or bend in order to fit within the outer tube 106. The dissection spatula 104 and hollow rod 112 may be made of a flexible material such as superelastic alloys (for example, nitinol), spring steel, or a flexible polymer.

The dissection spatula 104 may be curved in one or more section planes. For example, the dissection spatula 104 may be curved in a plane parallel to the longitudinal direction 114a and the vertical direction 114c. In use the concave side of the dissection spatula may face away from the retina. The curvature of the dissection spatula 104 may facilitate bending of the dissection spatula 104 thereby reducing pressure on the retina.

The dissection spatula 104 defines a channel 116. The channel 116 passes through the dissection spatula 104 from a point of attachment between the dissection spatula 104 and the hollow rod 112 to an opening 118 proximate a rounded distal edge 120 of the dissection spatula 104. The opening 118 may be formed in the edge 120 itself or may be proximate to the edge 120, e.g., within 0.1, 0.01, or 0.001 millimeters (mm). The hollow rod 112 may be hollow such that a continuous channel is defined between the hollow rod 112 and the channel 116 defined by the dissection spatula 104. For example, the hollow rod 112 and dissection spatula 104 may be monolithically formed such that the channel 116 within the dissection spatula 104 and the interior of the hollow rod 112 are a single channel.

The channel of the hollow rod 112 may be in fluid communication with a reservoir 122 of fluid. In the illustrated implementation, the reservoir 122 is housed within the handpiece 102. The illustrated reservoir is exemplary only and other arrangements are possible, such as a reservoir that is separate from the handpiece 102 and connected to the handpiece 102 by a tube. The reservoir 122 may be coupled to a pump 124, such as a mechanically, pneumatically, or electrically actuated pump. The pump 124 may be controlled by the button 110, such as the button 110 mechanically actuating the pump 124 in response to depression by a finger of the surgeon. In other embodiments, where the pump 124 is connected to or is incorporated into a surgical console, the pump 124 may be controlled by a footswitch in wired or wireless communication with the surgical console. For example, the footswitch may cause actuation of the pump 124 in response to depression by a foot of the surgeon. The pump 124 may be coupled to the hollow rod 112, such as by a tube 126. Accordingly, in response to actuation of the button 110, fluid from the reservoir 122 may be pumped out through the tube 126, hollow rod 112, and channel 116 to the opening 118.

The button 110, reservoir 122, pump 124, and tube 126 may be collectively considered to be a dispensing system for supplying fluid to the hollow rod 112. It shall be understood that there may be various implementations of a dispensing system that may be coupled to the hollow rod 112, including those in which the button 110 is omitted from the handpiece 102 and is on a remote device, such as a foot pedal, connected to the handpiece 102 by a tube. Likewise, the button 110 may be viewed as a control structure that may be substituted with a lever, diaphragm, touch-sensitive electronic component, or other component that may receive interaction from a surgeon in order to invoke pumping of fluid into the hollow rod 112.

Referring to FIG. 1B, in some embodiments, one or more teeth 104a are secured to or formed on the distal end of the dissection spatula 104. The teeth 104a may be angled such that outward facing surfaces (facing away from the dissection spatula 104) of the teeth 104a define an angle 104b with respect to an upper surface of the dissection spatula 104. For example, the angle 104b may be between 20 and 40 degrees, between 25 and 35 degrees, or between 29 and 31 degrees. For example, an angle 104b of 30 degrees has been found to be effective. In certain embodiments, the teeth 104a may have a length (e.g., height) from the upper side of the dissection spatula 104 between about 1 micrometer (μm) and about 20 μm, such as a length between about 1 μm and about 10 μm. The teeth may be used to scrape the membrane 200 (e.g., the ILM) off the retina 202, before the dissection spatula 104 is turned (e.g., rotated) for delamination of the membrane 200 from the retina 202. In certain embodiments, the teeth 104a may be sized and angled to limit the amount of penetration of edge 120.

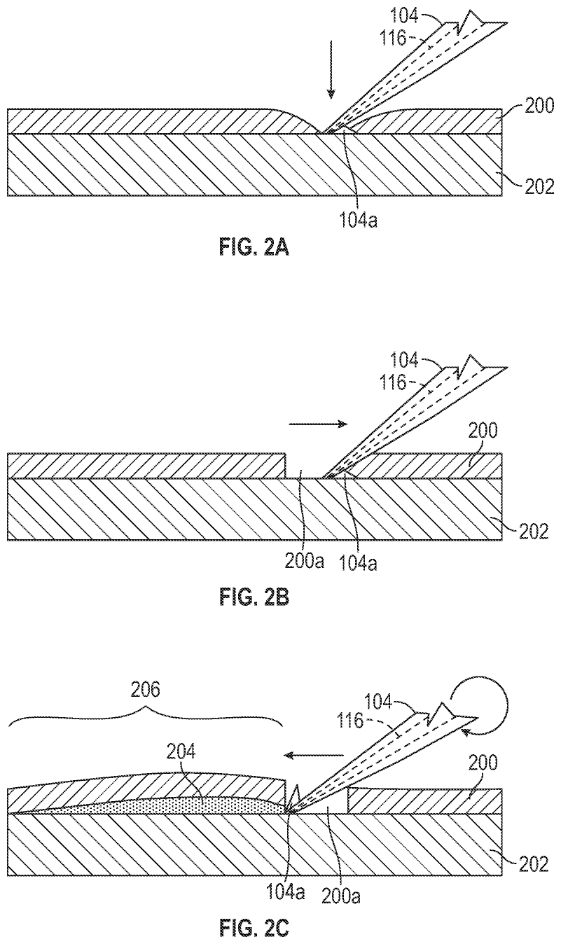

FIGS. 2A-2C illustrate the dissection spatula 104, with teeth 104a, during use. The dissection spatula 104 is first inserted through a trocar cannula in the patient's eye. When the outer tube 106 is being inserted through the trocar cannula, the dissection spatula 104 may be withdrawn within the outer tube 106. Once the distal end of the outer tube 106 is inserted into the eye, the dissection spatula 104 may then be extended relative to the outer tube 106 and brought into contact with a membrane 200 formed over the retina 202.

As shown in FIG. 2A, initially the dissection spatula 104 is pressed against the membrane 200 with the upper surface of the dissection spatula 104 facing the membrane 200. Accordingly, in the embodiments of FIGS. 2A-2C, the teeth 104a on the upper surface of the dissection spatula 104 are pressed into the membrane 200 such that they penetrate into the membrane 200. In such embodiments, the extent (i.e., the length) of the teeth 104a from the upper surface of the dissection spatula 104 and the angle 104b may be selected such that the teeth 104a do not penetrate the underlying retina 202 when pressed against the membrane 200. For example, the teeth 104a may extend outwardly from the upper surface by 10 microns or less. Alternatively, where teeth 104a are omitted or not used, indentation may be performed whereby the edge 120 cuts through the membrane 200. The width and rounded shape of the edge 120 may facilitate cutting of the membrane 200 without causing damage to the retina 202.

In FIG. 2B, the dissection spatula 104 is pulled across the membrane 200 to scrape the membrane 200 and create an opening 200a therein. For example, the teeth 104a on the upper surface of the dissection spatula 104 may be pulled toward the acute subtended angle defined between the dissection spatula 104 and the membrane 200 (to the right in FIG. 2A) to scrape the membrane 200 and form opening 200a. Stated differently, the teeth 104a may be pulled in the direction that the distal ends of the teeth 104a are pointing to form opening 200a. Note that opening 200a in FIG. 2B and FIG. 2C is exaggerated for clarity.

In FIG. 2C, the dissection spatula 104 is then rotated such that the teeth 104a are pointing away from the retina 202 and the lower surface of the dissection spatula 104 (the surface opposite the upper surface) is facing the membrane 200. The dissection spatula 104 may then be pushed through the opening 200a in a direction opposite to the pulling motion in FIG. 2B. Thereafter, the surgeon may actuate the button 110 in order to dispense fluid 204 from the reservoir 122 between the membrane 200 and the retina 202, thereby delaminating a portion 206 of the membrane 200 from the retina 202. The fluid 204 may be dyed, e.g., dyed blue, in order to enhance visibility of the portion of the membrane 200 that has been delaminated. The fluid 204 may be substantially inert, e.g., a saline solution.

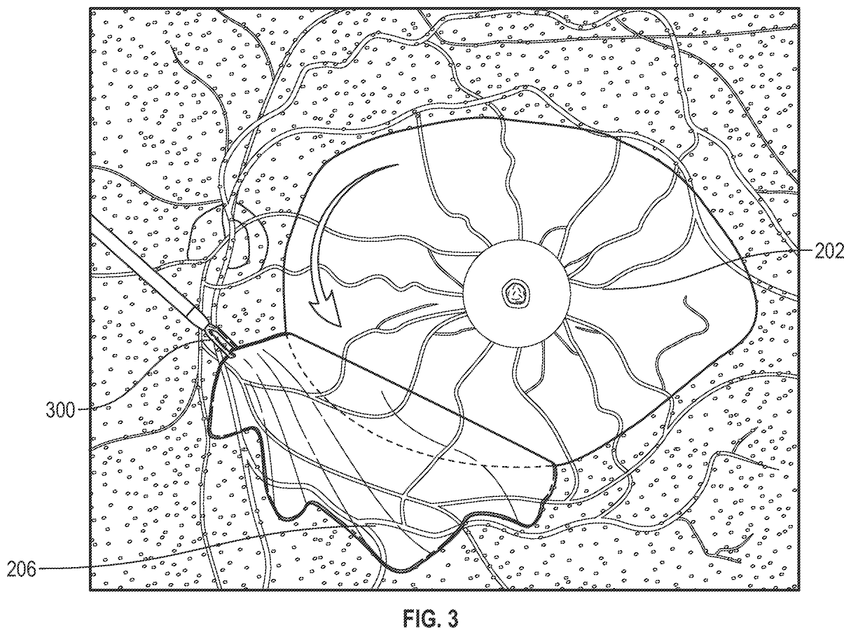

Referring to FIG. 3, following delamination, the dissection spatula 104 may be withdrawn into the outer tube 106 and the outer tube 106 may be withdrawn through the trocar cannula. Forceps 300 may then be inserted through the trocar cannula and used to grasp the portion of the membrane 200, such as adjacent the area where indentation occurred, and peel the membrane 200 using a circular motion. By injecting the fluid 204 between the membrane 200 and the retina 202 to delaminate the portion 206, the area to be peeled is more readily identifiable by the surgeon during the procedure, and the act of peeling is made easier with reduced traction of the portion 206 to the underlying retina 202. Accordingly, the dissection spatula 104 provides improved efficiency and safety as compared to more conventional devices used for membrane peeling.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

What is claimed is:

1. An ophthalmic surgical instrument for delaminating a retinal membrane, comprising:
a handpiece;
an actuator mounted on the handpiece;
an outer tube having a proximal end mounted to the handpiece;
a dissection spatula extendable outwardly relative to a distal end of the outer tube responsive to movement of the actuator, the dissection spatula defining a channel connected to an opening proximate a distal edge of the dissection spatula; and
a dispensing system in fluid communication with the channel.

2. The ophthalmic surgical instrument of claim 1, wherein the actuator is configured to move the outer tube relative to the handpiece.

3. The ophthalmic surgical instrument of claim 1, wherein the dissection spatula is wider than an inner diameter of the outer tube.

4. The ophthalmic surgical instrument of claim 1, wherein the dissection spatula comprises nitinol.

5. The ophthalmic surgical instrument of claim 1, further comprising a hollow rod connecting the dissection spatula to the handpiece, the hollow rod being in fluid communication with the channel and the dispensing system.

6. The ophthalmic surgical instrument of claim 1, wherein dispensing system includes one of a button mounted to the handpiece and a footswitch configured to cause fluid to flow through the channel.

7. The ophthalmic surgical instrument of claim 1, wherein the dispensing system includes a reservoir within the handpiece.

8. The ophthalmic surgical instrument of claim 1, wherein the outer tube defines a longitudinal direction that is collinear with an axis of symmetry of the outer tube, the dissection spatula being at least 2 times wider in a transverse direction perpendicular to the longitudinal direction than a thickness of the dissection spatula perpendicular to the transverse direction.

9. The ophthalmic surgical instrument of claim 8, wherein the dissection spatula is curved in a plane parallel to the longitudinal direction and a vertical direction that is perpendicular to the longitudinal direction and the transverse direction.

10. A method for peeling a membrane from a retina, the method comprising:
indenting the membrane with a distal edge of a dissection spatula; and
dispensing fluid from proximate the distal edge between the membrane and the retina to delaminate a portion of the membrane from the retina.

11. The method of claim 10, further comprising removing the portion of the membrane from the retina.

12. The method of claim 10, wherein the dissection spatula defines a channel in fluid communication with an opening proximate the distal edge.

13. The method of claim 12, wherein the dissection spatula is part of an ophthalmic instrument comprising:
a handpiece;
an actuator mounted on the handpiece; and
an outer tube having a proximal end mounted to the handpiece, the dissection spatula being extendable outwardly relative to a distal end of the outer tube responsive to movement of the actuator; and
a dispensing system in fluid communication with the channel.

14. The method of claim 13, further comprising dispensing the fluid responsive to interaction with a control element of the dispensing system.

15. The method of claim 13, wherein dispensing the fluid comprises dispensing the fluid from a reservoir within the handpiece.
